(19) United States
(12) Patent Application Publication
(10) Pub. No.: US 2026/0076659 A1
(43) Pub. Date: Mar. 19, 2026

(54) TREATMENT FOR GASTROINTESTINAL CAVITIES AND FISTULA

(71) Applicant: BOSTON SCIENTIFIC SCIMED, INC., Maple Grove, MN (US)

(72) Inventors: Martyn G. Folan, Galway (IE); Fergal Horgan, Galway (IE)

(73) Assignee: BOSTON SCIENTIFIC SCIMED, INC., Maple Grove, MN (US)

(21) Appl. No.: 19/328,834

(22) Filed: Sep. 15, 2025

Related U.S. Application Data
(60) Provisional application No. 63/695,089, filed on Sep. 16, 2024.

(51) Int. Cl. A61B 17/00

(52) U.S. Cl. CPC A61B 17/0057; A61B 2017/00557; A61B 2017/00592; A61B 2017/00597; A61B 2017/0061; A61B 2017/00623; A61B 2017/00641; A61B 2017/00659; A61B 2017/00818; A61B 2017/00862; A61B 2017/00951

(57) ABSTRACT

An implantable medical device may be implanted within a body lumen such as an esophagus in order to treat a wound within the body lumen. An esophagus has an inactive profile when the patient is not swallowing and an active profile when the patient is swallowing. The implantable medical device includes a barrier member that is adapted to radially expand and contract with the esophagus as the esophagus moves between the inactive profile and the active profile. An adhesive pattern is disposed on an outer surface of the barrier member and is adapted to adhere the barrier member to the esophagus such that the barrier member radially expands when the esophagus in in the active profile and radially contracts when the esophagus in in the inactive profile. The implantable medical device may be implanted using an expandable element such as an inflatable balloon.

### CROSS-REFERENCE TO RELATED APPLICATIONS

The application claims the benefit of U.S. Provisional Patent Application Ser. No. 63/695,089, filed on Sep. 16, 2024, the disclosure of which is incorporated herein by reference.

### TECHNICAL FIELD

The present disclosure relates generally to methods and apparatuses for various digestive ailments. More particularly, the disclosure relates to different configurations and methods of manufacture and use of an implant for treating esophageal wounds.

### BACKGROUND

Implants are placed in a body structure, such as a blood vessel, esophagus, trachea, biliary tract, colon, intestine, stomach or body cavity, to help treat wounds within the body structure. These devices are manufactured by any one of a variety of different manufacturing methods and may be used according to any one of a variety of methods. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

The disclosure is directed to several alternative designs, materials and methods of manufacturing medical device structures and assemblies, and the use thereof. An example may be found in an implantable medical device for implantation within a patient's esophagus, where the esophagus has an inactive profile when the patient is not swallowing and an active profile when the patient is swallowing. The implantable medical device includes a barrier member that is adapted to radially expand and contract with the esophagus as the esophagus moves between the inactive profile and the active profile. An adhesive pattern is disposed on an outer surface of the barrier member that is adapted to adhere the barrier member to the esophagus such that the barrier member radially expands when the esophagus in in the active profile and radially contracts when the esophagus in in the inactive profile.

Alternatively or additionally, the barrier member may be adapted to radially expand into a substantially circular cross-sectional profile when the esophagus is in the active pattern.

Alternatively or additionally, the barrier member may be adapted to radially contract into a star-shaped cross-sectional profile when the esophagus is in the inactive pattern.

Alternatively or additionally, the adhesive pattern may include a plurality of axially extending adhesive strips.

Alternatively or additionally, the adhesive pattern may include one or more circumferentially extending adhesive strips.

Alternatively or additionally, the barrier member may include an impermeable membrane.

Alternatively or additionally, the barrier member may include a silicone membrane or a polyurethane membrane.

Alternatively or additionally, the barrier member may include a tissue matrix.

Alternatively or additionally, the barrier member may include an inelastic inner layer, an elastic outer layer and a reflowable gel material that is disposed between the inner layer and the outer layer.

Another example may be found in a kit for treating a wound within a body lumen having an inner surface. The kit includes an implant having a collapsed configuration and an expanded configuration. The implant includes a barrier member having an outer surface and an adhesive pattern that is disposed on the outer surface of the barrier member. The kit includes an expandable delivery element for delivering the implant. The expandable delivery element is adapted to deliver the implant while the expandable delivery element and the implant are in a collapsed configuration and to expand to cause the body lumen and the implant to expand, causing the adhesive pattern to contact the inner surface of the body lumen.

Alternatively or additionally, the expandable delivery element may include an inflatable balloon.

Alternatively or additionally, the expandable delivery element may include an inflatable balloon having a plurality of balloon folds when the inflatable balloon is deflated.

Alternatively or additionally, the barrier member may be adapted to fold with the inflatable balloon such that the barrier member forms folds complementary to the balloon folds.

Alternatively or additionally, the adhesive pattern may include an adhesive that is activated only after a period of time post-delivery.

Alternatively or additionally, the kit may further include a sheath that is adapted to extend over the implant when the implant is in the collapsed configuration and to be withdrawn from the implant prior to expanding the expandable delivery element.

Another example may be found in an implantable medical device for implantation within a patient's esophagus, where the esophagus has an inactive profile when the patient is not swallowing and an active profile when the patient is swallowing. The implantable medical device includes a barrier member that is adapted to radially expand and contract with the esophagus as the esophagus moves between the inactive profile and the active profile. An adhesive pattern is disposed on an outer surface of the barrier member that is adapted to adhere the barrier member to the esophagus such that the barrier member radially expands when the esophagus in in the active profile and radially contracts when the esophagus in in the inactive profile.

Alternatively or additionally, the barrier member may be adapted to radially expand into a substantially circular cross-sectional profile when the esophagus is in the active pattern.

Alternatively or additionally, the barrier member may be adapted to radially contract into a star-shaped cross-sectional profile when the esophagus is in the inactive pattern.

Alternatively or additionally, the adhesive pattern may include a plurality of axially extending adhesive strips.

Alternatively or additionally, the adhesive pattern may include one or more circumferentially extending adhesive strips.

Alternatively or additionally, the barrier member may include an impermeable membrane.

Alternatively or additionally, the barrier member may include a silicone membrane or a polyurethane membrane.

Alternatively or additionally, the barrier member may include a tissue matrix.

Alternatively or additionally, the barrier member may include an inner layer, an outer layer, and a gel material that is disposed between the inner layer and the outer layer.

Alternatively or additionally, the inner layer may be inelastic, the outer layer may be elastic, and the gel material may be reflowable.

Another example may be found in a kit for treating a wound within a body lumen having an inner surface. The kit includes an implant having a collapsed configuration and an expanded configuration. The implant includes a barrier member having an outer surface and an adhesive pattern that is disposed on the outer surface of the barrier member. The kit includes an expandable delivery element for delivering the implant. The expandable delivery element is adapted to deliver the implant while the expandable delivery element and the implant are in a collapsed configuration and to expand to cause the body lumen and the implant to expand, causing the adhesive pattern to contact the inner surface of the body lumen.

Alternatively or additionally, the expandable delivery element may include an inflatable balloon.

Alternatively or additionally, the expandable delivery element may include an inflatable balloon having a plurality of balloon folds when the inflatable balloon is deflated.

Alternatively or additionally, the barrier member may be adapted to fold with the inflatable balloon such that the barrier member forms folds that are complementary to the balloon folds.

Alternatively or additionally, the adhesive pattern may include an adhesive that is activated only after a period of time post-delivery.

Alternatively or additionally, the kit may further include a sheath that is adapted to extend over the implant when the implant is in the collapsed configuration and to be withdrawn from the implant prior to expanding the expandable delivery element.

Alternatively or additionally, the body lumen may include an esophagus.

Another example may be found in a method for treating a wound within a body lumen. The method includes advancing an assembly to the wound within the body lumen, the assembly including an inflatable balloon and an implant disposed on the inflatable balloon, the implant including a barrier member and an adhesive pattern disposed on the barrier member. The inflatable balloon is inflated in order to expand the barrier member and urge the adhesive pattern against an inner surface of the body lumen. The adhesive pattern is allowed to adhere to the inner surface of the body lumen. The inflatable balloon is deflated.

Alternatively or additionally, inflating the inflatable balloon may also expand the body lumen. Deflating the inflatable balloon allows the body lumen to return to a non-expanded configuration.

Alternatively or additionally, the assembly may further include a removable sheath that extends over the implant. The method may further include withdrawing the removable sheath prior to inflating the inflatable balloon.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1A is a schematic cross-sectional view of an esophagus shown in an active profile, i.e., the patient is swallowing;

FIG. 1B is a schematic cross-sectional view of an esophagus shown in an inactive profile, i.e., the patient is not swallowing;

FIG. 2A is a schematic cross-sectional view of the esophagus of FIG. 1A, shown in the active profile with an illustrative implant secured within the esophagus;

FIG. 2B is a schematic cross-sectional view of the esophagus of FIG. 1A, shown in the inactive profile with the illustrative implant secured within the esophagus;

FIG. 3 is a perspective view of an illustrative implantable medical device (implant) that may be used as the illustrative implant shown in FIGS. 2A and 2B;

FIG. 4 is a perspective view of the illustrative implantable medical device of FIG. 3 shown in combination with an inflatable balloon for deploying the illustrative implantable medical device;

FIGS. 5A, 5B, 5C and 5D are schematic views showing a step-by-step process for implanting the illustrative implantable medical device of FIG. 3;

FIGS. 6A, 6B, 6C and 6D are schematic views showing example uses for the illustrative implantable medical device of FIG. 3 in conjunction with ancillary devices;

FIG. 7 is a perspective view of an illustrative implantable medical device (implant) that may be used as the illustrative implant shown in FIGS. 2A and 2B;

FIG. 8 is a cross-sectional view taken along line 8-8 of FIG. 7;

FIG. 9 is a cross-sectional view taken along line 9-9 of FIG. 7;

FIG. 10 is a schematic view of the illustrative implantable medical device of FIG. 7, shown implanted within a body lumen proximate a fistula;

FIG. 11A is a schematic cross-sectional view of the implantable medical device and inflatable balloon of FIG. 4, shown with the inflatable balloon deflated and partially folded;

FIG. 11B is a schematic cross-sectional view of the implantable medical device and inflatable balloon of FIG. 4, shown with the inflatable balloon deflated and completely folded;

FIG. 12A is a schematic view showing an illustrative implantable medical device disposed over an expandable delivery device, with the expandable delivery device compressed for delivery;

FIG. 12B is a schematic view showing the illustrative implantable medical device of FIG. 12A, with the expandable delivery device expanded;

FIG. 13A is a schematic view showing an illustrative implantable medical device disposed within an illustrative delivery device, with a removeable sheath disposed over the illustrative implantable medical device;

FIG. 13B is a schematic view showing the illustrative implantable medical device and illustrative delivery device of FIG. 13A, shown with the removeable sheath withdrawn proximally;

FIG. 13C is a schematic view showing the illustrative implantable medical device and illustrative delivery device of FIG. 13B, with an inflatable balloon inflated; and

FIG. 13D is a schematic view showing the illustrative implantable medical device and illustrative delivery device of FIG. 13C, with the inflatable balloon deflated.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or”unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

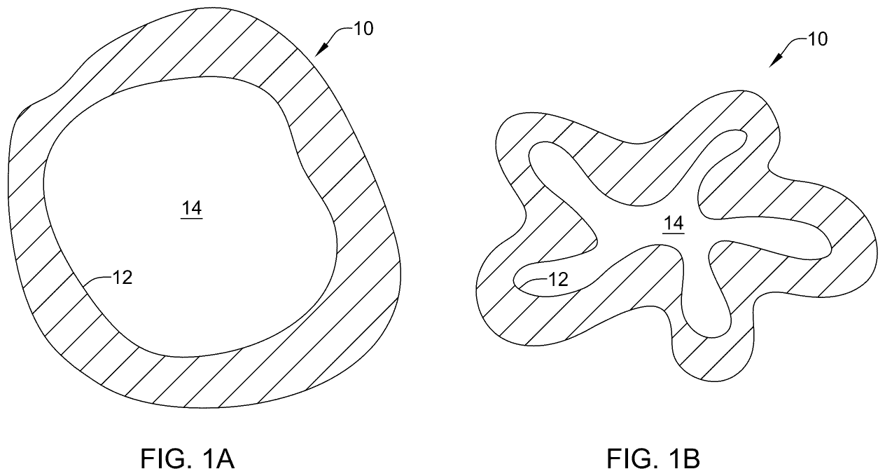

The esophagus is a fibromuscular organ that allows food passage, aided by peristaltic contracts, from the pharynx to the stomach. The esophagus passes behind the trachea and heart, through the diaphragm, and empties into the uppermost region of the stomach. The esophagus and the trachea share a common anatomical space such that either the esophagus or the trachea, depending on its own requirements, can expand to occupy a majority of the volume within the common anatomical space. When a person is breathing, the trachea may expand. When a person is swallowing, the esophagus may expand. FIG. 1A is a schematic cross-sectional view through an esophagus 10 with the esophagus 10 shown in an active profile, meaning that the person is in the process of swallowing a food bolus (not shown). An inner surface 12 of the esophagus 10 defines a passage 14 that extends through the esophagus 10.

In some cases, the esophagus 10 may be considered as having a substantially circular cross-sectional profile when in the active profile. Substantially circular may be defined in terms of having a first diameter extending across the cross-sectional profile and a second diameter that is orthogonal to the first diameter and also extends across the cross-sectional profile, where the second diameter is plus or minus within about twenty percent or less of the first diameter, or the second diameter is plus or minus within about ten percent or less of the first diameter.

FIG. 1B is a schematic cross-sectional view through the esophagus 10 with the esophagus 10 shown in an inactive profile. The inactive profile corresponds to when the person is not swallowing. As a result, the esophagus 10 collapses into a star shape in which the esophagus 10 consumes relatively less of the volume within the common anatomical space shared by the esophagus 10 and the trachea (not shown). It will be appreciated that the inactive profile of the esophagus 10 may be considered as being a resting configuration for the esophagus 10, as a person only eats and swallows periodically, but the person breathes all of the time. At rest, a person may breath perhaps every five or ten seconds, or even every fifteen seconds. During activity, a person may breath more frequently. It will be appreciated that FIGS. 1A and 1B may not be to scale with respect to each other.

The esophagus 10 may develop a wound such as a perforation, anastomosis or a fistula for a variety of reasons, such as but not limited to insufficiency in post-surgical sutures, spontaneous perforation (such as Boerhaave syndrome), volatile ingestion such as swallowing a sold poorly masticated food bolus or a sharp object or caustic substances, and congenital conditions such as pediatric esophageal atresia. Similar wounds may occur in a variety of different body lumens, but for simplicity the invention will be discussed with respect to treating a wound within the esophagus 10. While in some cases, this has been treated by implanting a fully covered self-expanding stent, it will be appreciated that the radial forces imparted on the esophagus 10 can tend to hold at least a portion of the esophagus 10 open as shown in FIG. 1A, not allowing the esophagus 10 to retract into its inactive profile as shown in FIG. 1B.

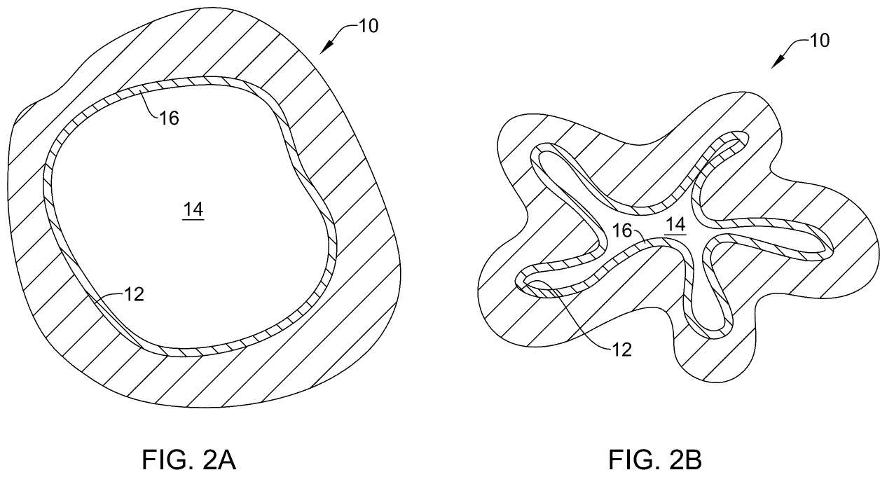

In some instances, a flexible implant may be implanted into the esophagus 10 proximate a wound. The flexible implant may be adhered to the inner surface 12 while the esophagus 10 is in its active profile and yet remain adhered to the inner surface 12 even when the esophagus 10 reverts to its inactive profile. This may be seen in FIGS. 2A and 2B, in which an implant 16 may be seen as being secured relative to the inner surface 12 of the esophagus 10. FIG. 2A shows the esophagus 10 (and hence the implant 16) in the active profile and FIG. 2B shows the esophagus 10 (and hence the implant 16) in the inactive profile. It will be appreciated that the implant 16 is able to move radially as the esophagus 10 moves radially. As a result, the implant 16 does not interfere with the esophagus 10 being able to move from the inactive profile, to the active profile as the person swallows, and back to the inactive profile.

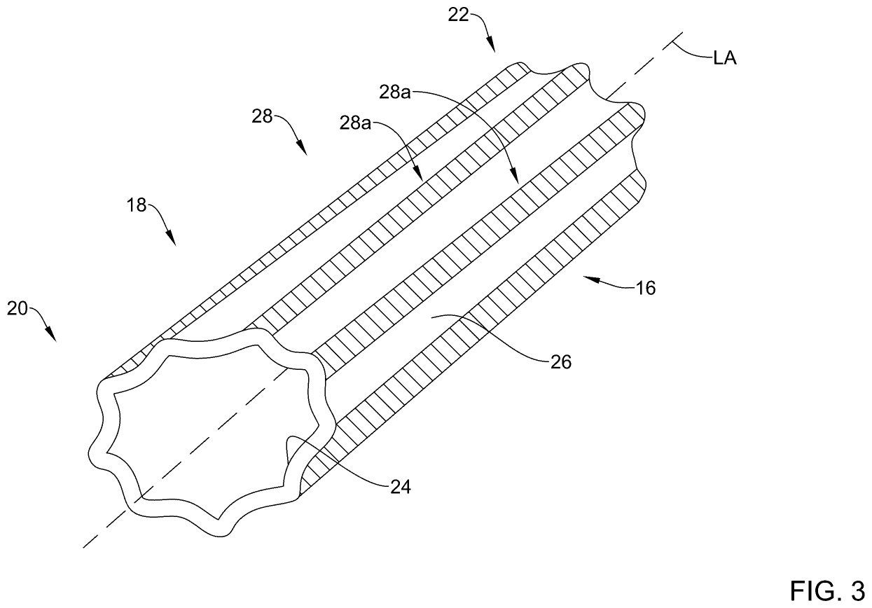

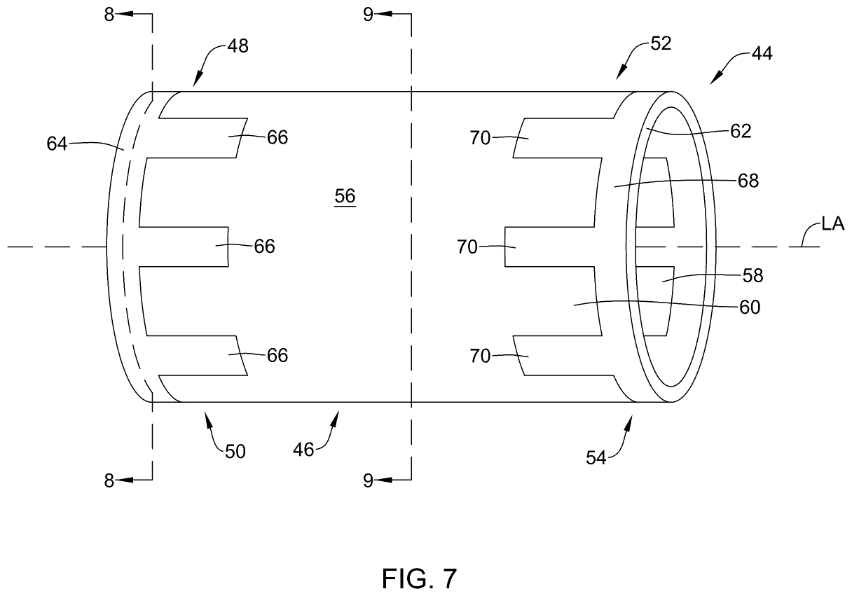

FIG. 3 is a perspective view of the implant 16. The implant 16 includes a barrier member 18 that is adapted to radially expand and contract with the esophagus 10 as the esophagus 10 moves between the inactive profile and the active profile. In some cases, the barrier member 18 may include an impermeable membrane. In some cases, the barrier member 18 may include a silicone membrane such as that commercially available under the Permalume™ name or a polyurethane membrane such as those commercially available under the ChronoFlex® and Carbothane™ tradenames. The barrier member 18 may include a silicone polycarbonate polyurethane such as those commercially available under the Carbosil® tradename. In some cases, the barrier member 18 may be a more complex structure, as will be discussed with respect to FIGS. 7 through 10.

The implant 16 may be dimensioned in accordance with the specific anatomy in which the implant 16 may be implanted. For use in an adult patient's esophagus 10, for example, the implant 16 may have an overall length ranging from 3 centimeters (cm) to 10 cm and an overall diameter (when expanded) that ranges from 14 millimeters (mm) to 30 mm. For use in other body lumens, the implant 16 may have an overall length ranging from 2 cm to 10 cm and an overall diameter (when expanded) that ranges from 10 mm to 50 mm. Other suitable body lumens include body lumens in the gastric region such as the pylorus, duodenum, biliary ducts, colon, rectum, trachea, brochus, gastric sleeve (post bariatric surgery) and gastric pouch (post Roux-en-Y surgery).

In some cases, the barrier member 18 may include a tissue matrix. The barrier member 18 may include an allogenic and non-immunogenic tissue matrix that is grown from human donor cells. The barrier member 18 may include a regenerative acellular “allograft” tissue matrix. As an example, the dimensionally controlled tissue-engineered barrier member 18 may be grown from neonatal human dermal fibroblasts that are entrapped in bovine fibrin gel, prior to being decellularized. Using a biological material allows for the barrier member 18 to become the patient's own living, growing tissue. In some cases, the barrier member 18 would be created in a set shape or geometry, and would be a tube having controlled mechanical properties and controlled thickness in a single layer material. In some cases, particularly when treating a fistula, the barrier member 18 may promote recellularization post implantation, won't induce any immune response, and will not contribute to thrombus risk.

The barrier member 18 has a longitudinal axis LA, and extends from a first end 20 to a second end 22. In some cases, the first end 20 may be considered as being a distal end and the second end 22 may be considered as being a proximal end, for example. The barrier member 18 has an inner surface 24 and an outer surface 26. An adhesive pattern 28 that includes a number of adhesive stripes 28a is disposed on the outer surface 26 of the barrier member 18. In some cases, the adhesive pattern 28 may be adapted to adhere the barrier member 18 to the inner surface 12 of the esophagus 10 such that the barrier member 18 will radially expand when the esophagus 10 radially expands into the active profile and to radially contract when the esophagus 10 radially contracts into the inactive profile. In some cases, at least some of the adhesive stripes 28a extend parallel or at least substantially parallel with the longitudinal axis LA. Substantially parallel may be defined as within about twenty percent or less of parallel, or within about ten percent or less of parallel. The adhesive stripes 28a may be applied to the outer surface 26 of the barrier member 18 using any suitable technique, including spraying and painting. In some cases, the entire outer surface 26 of the barrier member 18 may be covered by adhesive.

In some cases, the adhesive pattern 28 may be formed of a mucosal adhesive substance that can adhesively bind to the inner surface 12 of the esophagus 10 (or bind an interior surface of other body lumens). As an example, the adhesive pattern 28 may be formed of a combination of a gel named ChitoSpace that is made from chitosan, PEG SG (succinimidyl glutarate ester) and a pH 11 buffer. This two-part gel crosslinks due to pH changes as it neutralizes with the chitosan in an acidic solution and the PEG SG as a result of the pH 11 buffer. Other adhesives may also be used. In some cases, the adhesive pattern 28 is formed of an adhesive that is not immediately adhesive, but becomes adhesive after a period of time in which the adhesive is exposed to bodily fluids, for example.

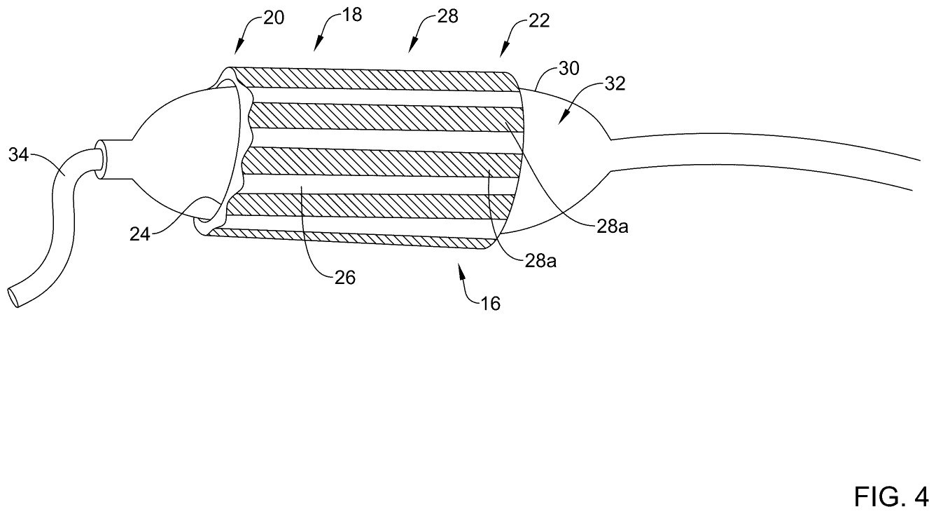

In some cases, the implant 16 may be expandable using an expandable element such as an expandable stent or an inflatable balloon. FIG. 4 shows the implant 16 positioned over an outer surface 30 of an inflatable balloon 32. The inflatable balloon 32 may be advanced over a guidewire 34. In some instances, the implant 16 and the inflatable balloon 32, or a delivery device including the inflatable balloon 32, may in combination be considered as a kit that may be used in treating a wound within the esophagus 10 or another body lumen (not shown).

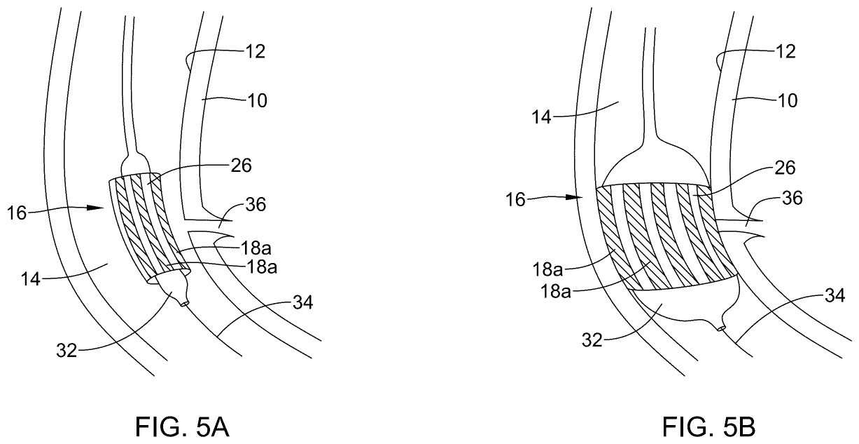

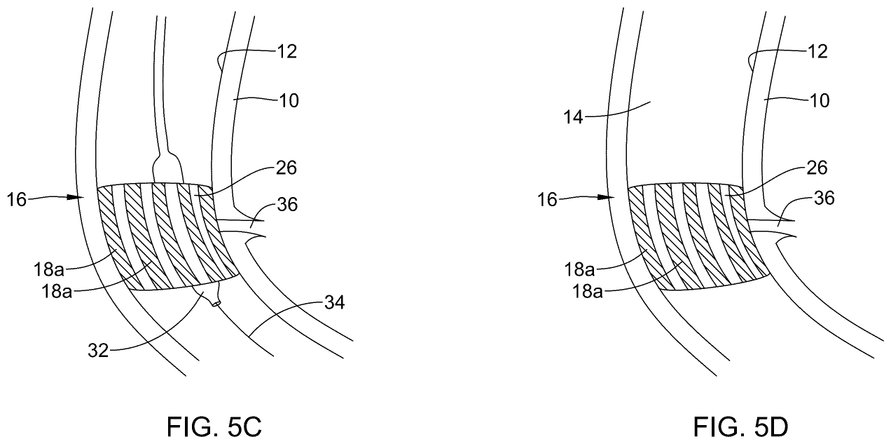

FIGS. 5A, 5B, 5C and 5D together show a step-by-step process for treating a wound within the esophagus 10 using the implant 16. In this example, the esophagus 10 includes a fistula 36. In some cases, the fistula 36 may extend into the trachea (not shown). In some cases, the fistula 36 may extend into another body feature or element. In FIG. 5A, the implant 16, disposed over the inflatable balloon 32, has been advanced through the esophagus 10 to a desired anatomical location, which in this case corresponds to positioning the implant 16 such that the implant 16 will extend across an opening of the fistula 36. In some cases, a sheath (not shown) may extend over the implant 16 in order to prevent the adhesive pattern 28 from premature adhering to something. The sheath, if included, may be withdrawn proximally prior to inflating the inflatable balloon 32. In FIG. 5B, the inflatable balloon 32 has been inflated to a diameter that forms an interference fit with the esophagus 10, and may help ensure that the esophagus 10 expands into its active profile. This expands the esophagus 10 and helps to ensure that the esophagus 10 reaches a more circular cross-sectional profile.

FIG. 5C shows the inflatable balloon 32 having been deflated after a period of time sufficient to allow the implant 16 to become adhesively secured to the inner surface 12 of the esophagus 10 via the adhesive pattern 28. How long this takes may depend at least in part upon the specific adhesive used. As an example, the inflatable balloon 32 may remain inflated, in order to urge the adhesive pattern 28 into contact with the inner surface 12 of the esophagus 10 for a period of time ranging from about ten seconds to about sixty seconds, or perhaps twenty to forty seconds, for example. FIG. 5D shows the implant 16 secured within the esophagus 10 after the inflatable balloon 32 and the guidewire 34 have been removed. The esophagus 10 is then able to revert back to its inactive profile.

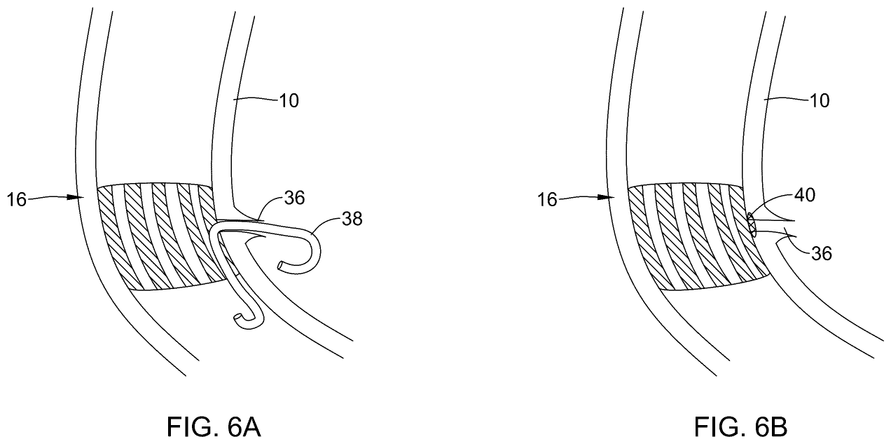

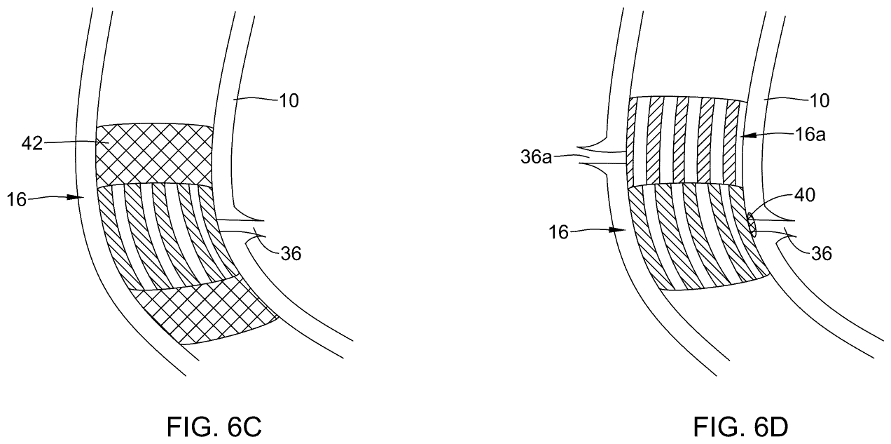

FIGS. 6A, 6B, 6C and 6D provide additional examples of how the implant 16 may be utilized in treating a wound (such as the fistula 36) within the esophagus 10 in conjunction with ancillary treatment options. In FIG. 6A, a plastic drainage stent 38 extends through the fistula 36. The implant 16 may be positioned to help hold the plastic drainage stent 38 in position relative to the fistula 36 so that the plastic drainage stent 38 is less likely to migrate or be dislodged. The plastic drainage stent 38 permits drainage while the implant 16 holds the plastic drainage stent 38 in position. In FIG. 6B, several clips 40 are positioned relative to the fistula 36. The clips 40 may be metal clips, for example, or may be plastic clips. The implant 16 holds the clips 40 in position so that they are less likely to migrate or be dislodged. In some cases, as shown in FIG. 6C, the implant 16 may be used in combination with a self-expanding stent 42 in cases in which additional isolation protection may be desired.

In FIG. 6D, the implant 16 is shown positioned such that it extends over the fistula 36. A second implant 16a is positioned adjacent the first implant 16, and is positioned such that the second implant 16a spans a second fistula 36a. In some cases, the first implant 16 and the second implant 16a may be positioned such that one of the first implant 16 and the second implant 16a overlaps part of the other of the first implant 16 and the second implant 16a. In some cases, as shown, the first implant 16 and the second implant 16a may be positioned end to end. In some cases, more than two implants 16 may be deployed within the esophagus 10, depending on the number and severity of the wounds within the esophagus 10. While not shown, in some cases, particularly when the fistula 36 extends from the esophagus 10 to the trachea (not shown), a first implant 16 may be positioned within the esophagus 10 such that the first implant 16 spans the esophagus end of the fistula 36 and a second implant 16 may be positioned within the trachea such that the second implant 16 spans the trachea end of the fistula 36.

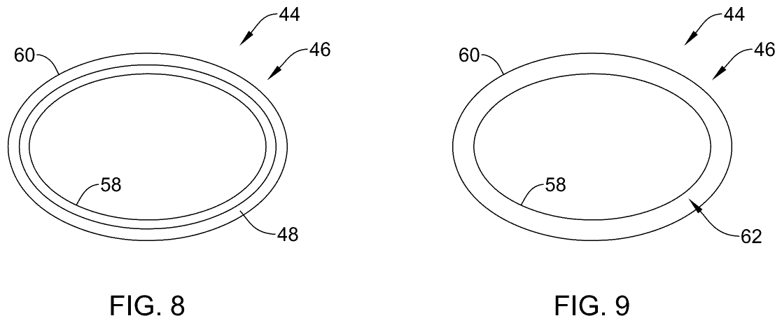

FIG. 7 is a perspective view of an illustrative implant 44 that may be used as the implant 16. FIG. 8 is a cross-sectional view taken along the line 8-8 of FIG. 7 and FIG. 9 is a cross-sectional view taken along the line 9-9 of FIG. 7. The implant 44 includes a barrier member 46, a first adhesive pattern 48 disposed proximate a first end 50 of the implant 44 and a second adhesive pattern 52 that is disposed proximate a second end 54 of the implant 44. The barrier member 46 has an outer surface 56. In some cases, the first adhesive pattern 48 and/or the second adhesive pattern 52 may be disposed on the outer surface 56. In some cases, the first adhesive pattern 48 and/or the second adhesive pattern 52 may be at least partially set into the outer surface 56 such that the first adhesive pattern 48 and/or the second adhesive pattern 52 extend radially beyond the outer surface 56 a distance that is less than a thickness of the corresponding first adhesive pattern 48 and/or the second adhesive pattern 52. In some cases, an outer surface of the first adhesive pattern 48 and/or the second adhesive pattern 52 may be flush with the outer surface 56.

The implant 44 may be dimensioned in accordance with the specific anatomy in which the implant 44 may be implanted. For use in an adult patient's esophagus 10, for example, the implant 44 may have an overall length ranging from 3 centimeters (cm) to 10 cm and an overall diameter (when expanded) that ranges from 14 millimeters (mm) to 30 mm. For use in other body lumens, the implant 44 may have an overall length ranging from 2 cm to 10 cm and an overall diameter (when expanded) that ranges from 10 mm to 50 mm. Other suitable body lumens include body lumens in the gastric region such as the pylorus, duodenum, biliary ducts, colon, rectum, trachea, brochus, gastric sleeve (post bariatric surgery) and gastric pouch (post Roux-en-Y surgery).

In some cases, the barrier member 46 may include an inner layer 58, an outer layer 60 and a gel material 62 that is disposed between the inner layer 58 and the outer layer 60. In some cases, the inner layer 58 may be inelastic or at least relatively inelastic and the outer layer 60 may be elastic or at least relatively elastic. In some cases, the outer layer 60 may be considered as being more elastic than the inner layer 58, and the inner layer 58 may be considered as being less elastic than the outer layer 60. In some cases, the gel material 62 may be reflowable, particularly if pressure is applied to the gel material 62.

In some cases, the inner layer 58 may be made from polymers such as Chronoflex® 80A, which has a Shore durometer of 75 D within a pressure range of 600 to 3000 pounds per square inch (psi), Carbothane™ 90A, which has a Shore durometer of 70 D within a pressure range of 350 to 1050 psi, Carbosil® 90A, which has a Shore durometer of 55 D within a pressure range of 1400 to 2500 psi, and ePTFE (expanded polytetrafluoroethylene). In some cases, the outer layer 60 may be made of silicone. Natural materials for forming the gel material 62 may include hyaluronic acid, chitosan, heparan, alginate, gelatin and fibrin. Synthetic materials for forming the gel material 62 may include polyvinyl alcohol, polyethylene glycol (PEG), polyacrylate, sodium polyacrylate, acrylate polymers, polyvinylpyrrolidone (PVP) and polymers and copolymers thereof.

In some cases, the first adhesive pattern 48 may include a circumferentially extending adhesive strip 64 that may be considered as being at least substantially orthogonal to the longitudinal axis LA, where substantially orthogonal may be defined as within about twenty percent or less of orthogonal, or within about ten percent or less of orthogonal. The first adhesive pattern 48 may include several axially extending adhesive strips 66 that may be considered as being at least substantially parallel with the longitudinal axis LA, where substantially parallel may be defined as within about twenty percent or less of parallel, or within about ten percent or less of parallel. Similarly, the second adhesive pattern 52 may include a circumferentially extending adhesive strip 68 that may be considered as being at least substantially orthogonal to the longitudinal axis LA, where substantially orthogonal may be defined as within about twenty percent or less of orthogonal, or within about ten percent or less of orthogonal. The second adhesive pattern 52 may include several axially extending adhesive strips 70 that may be considered as being at least substantially parallel with the longitudinal axis LA, where substantially parallel may be defined as within about twenty percent or less of parallel, or within about ten percent or less of parallel.

In some cases, the first adhesive pattern 48 and/or the second adhesive pattern 52 may be formed of a mucosal adhesive substance that can adhesively bind to the inner surface 12 of the esophagus 10 (or bind an interior surface of other body lumens). As an example, the first adhesive pattern 48 and/or the second adhesive pattern 52 may be formed of a combination of a gel named ChitoSpace that is made from chitosan, PEG SG (succinimidyl glutarate ester) and a pH 11 buffer. This two-part gel crosslinks due to pH changes as it neutralizes with the chitosan in an acidic solution and the PEG SG as a result of the pH 11 buffer. Other adhesives may also be used. In some cases, the first adhesive pattern 48 and/or the second adhesive pattern 52 may be formed of an adhesive that is not immediately adhesive, but becomes adhesive after a period of time in which the adhesive is exposed to bodily fluids, for example.

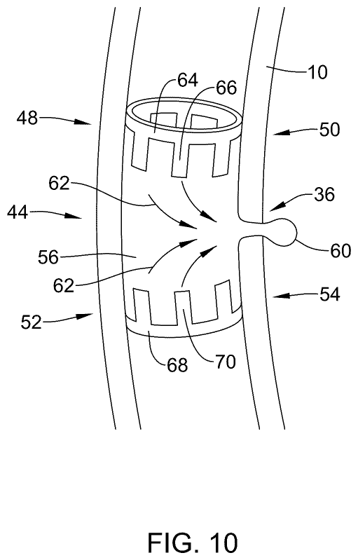

As noted, the gel material 62 may be adapted to reflow when pressure is applied. In some cases, expanding the implant 44 causes the implant 44 to form an interference fit with the esophagus 10. This can cause the gel material 62 to preferentially flow away from higher pressure locations (such as where the implant 44 interacts with the esophagus 10) towards lower pressure locations (such as where the opening of the fistula 36 is). FIG. 10 is a schematic view of the implant 44 disposed within the esophagus 10 such that the implant 44 extends over the fistula 36. Because the outer layer 60 is elastic, the outer layer 60 may be able to extend into and through the fistula 36. This allows the gel material 62 to also flow into and through the fistula 36, thereby accelerating closure of the fistula 36.

In some cases, the implant 16 and the implant 44 may be used in other anatomical locations where post-surgical and/or spontaneous leaks are to be expected. For example, the implant 16 and/or the implant 44 may be used to treat post bariatric surgical leaks where a sleeve gastrectomy has performed. The staple lines that result from a sleeve gastrectomy can be prone to leak, and can be a good application for either the implant 16 or the implant 44. The implant 16 and/or the implant 44 may be used to treat post bariatric surgical leaks where a Roux-en-Y surgery have been performed. Both ends of the Roux limb that results from this procedure can be prone to leak, and can be a good application for either the implant 16 or the implant 44. In some cases, given the high risk nature of these procedures, it may be useful to use the implant 16 and/or the implant 44 in a prophylactic capacity, implanting the implant 16 and/or the implant 44 at the time of primary surgery in order to reinforce damaged tissue areas. This may reduce the necessity of performing subsequent procedures to eliminate leaks.

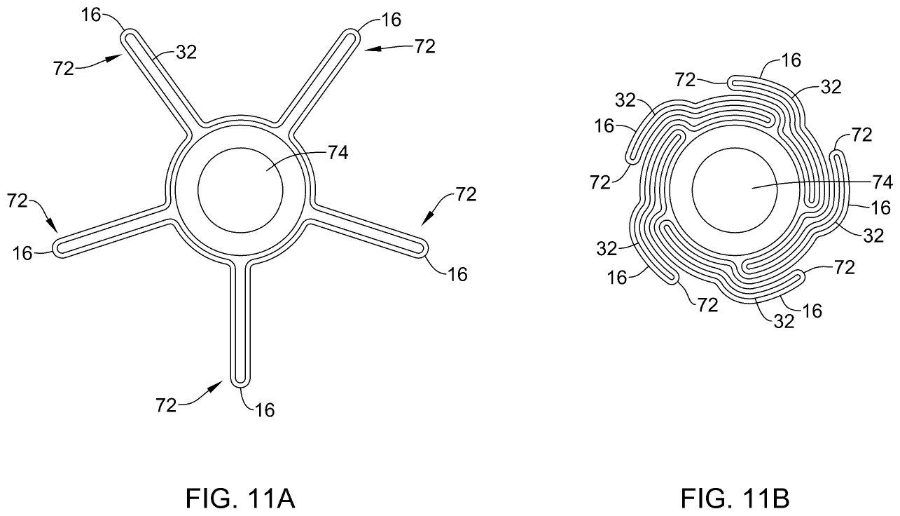

FIGS. 11A and 11B are schematic views of the implant 16 disposed over the inflatable balloon 32, showing how the implant 16 may be folded with the inflatable balloon 32. In FIG. 11A, the inflatable balloon 32 is deflated, and has been partially folded. This results in a number of balloon wings 72. A total of five balloon wings 72 are shown. In some cases, there may be four or fewer balloon wings 72. In some cases, there may be six or more balloon wings 72. As can be seen in FIG. 1B, the implant 16 becomes folded in combination with the inflatable balloon 32, which facilitates holding the implant 16 in position prior to inflating the inflatable balloon 32. As the inflatable balloon 32 is inflated within the esophagus 10, the implant 16 will expand as the inflatable balloon 32. This helps to press the implant 16 (and the adhesive pattern 28 formed thereon) into contact with the inner surface 12 of the esophagus 10, thereby helping to adhere the adhesive pattern 28 to the inner surface 12 of the esophagus 10. The inflatable balloon 32 is seen extending about a device lumen 74, such as an inflation lumen.

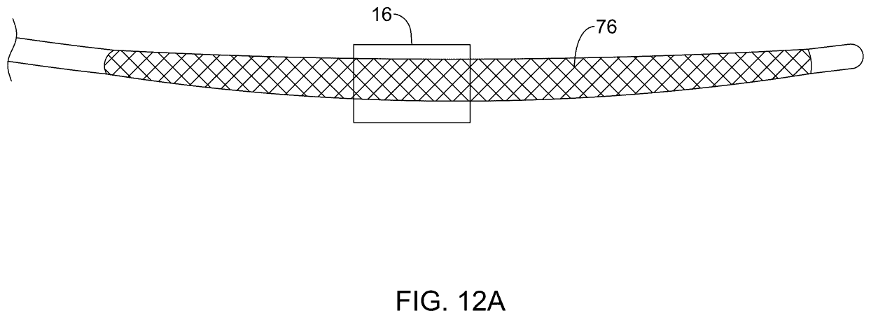

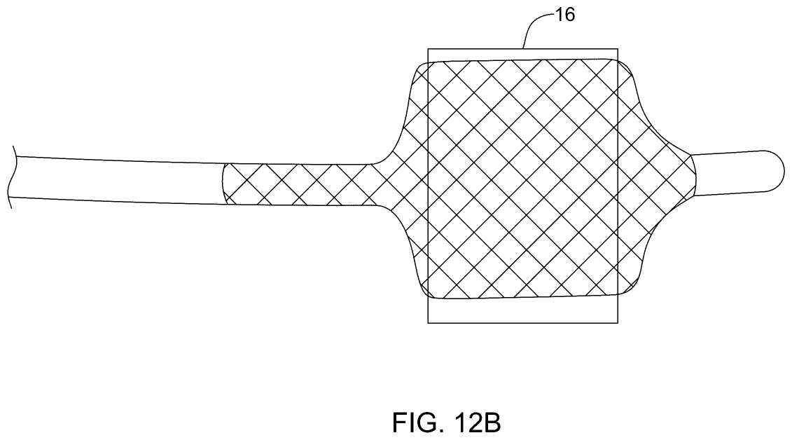

In some cases, the implant 16 (or the implant 44) may be expanded without using an inflatable balloon such as the inflatable balloon 32. In some cases, as shown in FIGS. 12A and 12B, the implant 16 (or the implant 44) may be disposed on an unexpanded braid. FIG. 12A shows the implant 16 disposed over an unexpanded braid 76. In FIG. 12B, the braid 76 has been expanded, thereby causing the implant 16 to expand as well. In some cases, the braid 76 may be expanded by causing the braid 76 to shorten. Accordingly, the braid 76 may be subsequently returned to its unexpanded configuration by causing the braid 76 to lengthen after the implant 16 has been expanded and has been held against the esophagus 10 long enough for the adhesive pattern 28 to adhere to the inner surface 12 of the esophagus 10. The braid 76 may also be used for delivering and deploying the implant 44.

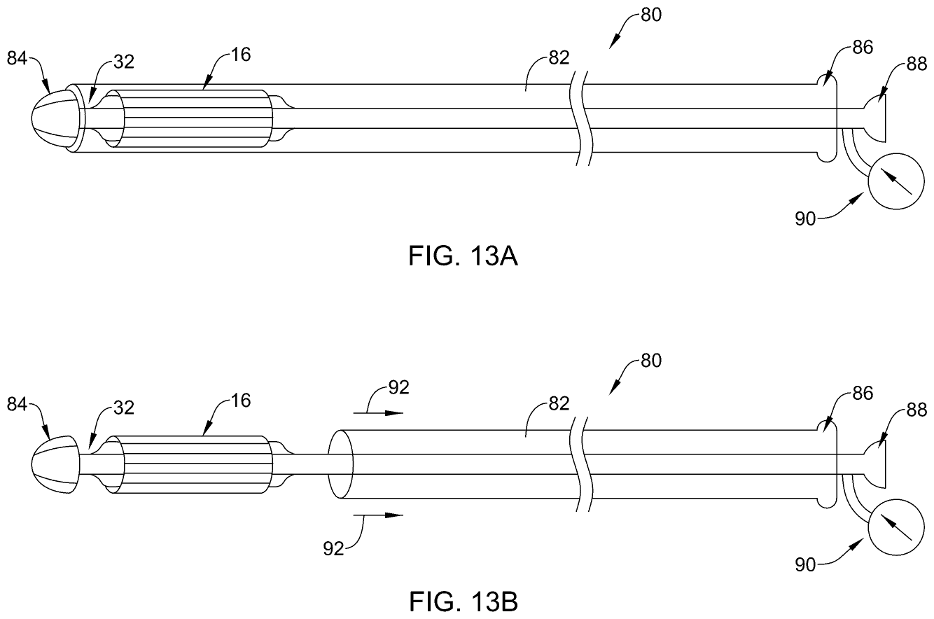

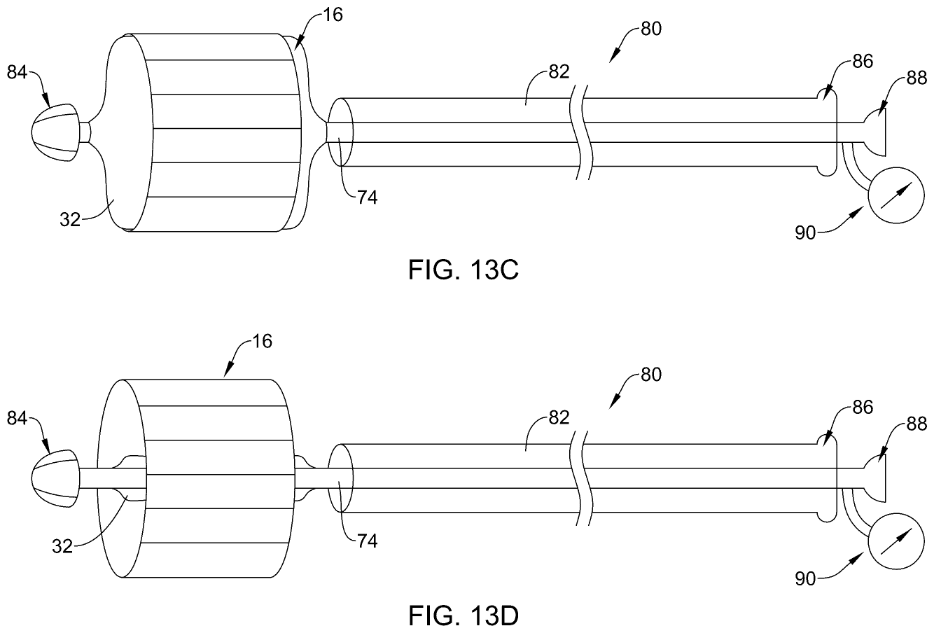

FIGS. 13A, 13B, 13C and 13D are schematic views showing the implant 16 within a delivery device 80. The delivery device 80 includes an outer sheath 82 that (as shown in FIG. 13A) extends over both the inflatable balloon 32 and the implant 16 that is disposed on the inflatable balloon 32. The outer sheath 82 is shown in FIG. 13A as being transparent so that the inflatable balloon 32 and the implant 16 may be seen. The delivery device 80 includes an outer control handle 86 and an inner control handle 88 that is arranged coaxially with respect to the outer control handle 86. The device 80 includes a balloon expansion mechanism 90 that may be used to inflate and deflate the inflatable balloon 32. Because the inflatable balloon 32 is deflated in FIG. 13A, the balloon expansion mechanism 90 shows zero pressure. In FIG. 13B, the outer sheath 82 has been withdrawn proximally, in a direction indicated by arrows 92.

In FIG. 13C, the inflatable balloon 32 has been inflated. The balloon expansion mechanism 90 now shows a positive pressure. The inflatable balloon 32 expands the implant 16 and may press the implant 16 into contact with the inner surface 12 of the esophagus 10 for long enough for the adhesive pattern 28 on the implant 16 to adhere to the inner surface 12 of the esophagus 10. After sufficient time, the inflatable balloon 32 may once again be deflated, as shown in FIG. 13D. The balloon expansion mechanism 90 once again shows zero pressure. The delivery device 80, in combination with the implant 16, may be considered as a kit for treating a wound within a body lumen such as the esophagus 10. While described with respect to the implant 16, the implant 44 may be implanted in a similar fashion as shown in FIGS. 13A, 13B, 13C and 13D.

The materials that can be used for the various components of the medical stent(s), and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent and/or elements or components thereof. In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. An implantable medical device for implantation within a patient's esophagus, the esophagus having an inactive profile when the patient is not swallowing and an active profile when the patient is swallowing, the implantable medical device comprising:
a barrier member adapted to radially expand and contract with the esophagus as the esophagus moves between the inactive profile and the active profile; and
an adhesive pattern disposed on an outer surface of the barrier member, the adhesive pattern adapted to adhere the barrier member to the esophagus such that the barrier member radially expands when the esophagus in in the active profile and radially contracts when the esophagus in in the inactive profile.

2. The implantable medical device of claim 1, wherein the barrier member is adapted to radially expand into a substantially circular cross-sectional profile when the esophagus is in the active pattern.

3. The implantable medical device of claim 1, wherein the barrier member is adapted to radially contract into a star-shaped cross-sectional profile when the esophagus is in the inactive pattern.

4. The implantable medical device of claim 1, wherein the adhesive pattern comprises a plurality of axially extending adhesive strips.

5. The implantable medical device of claim 1, wherein the adhesive pattern comprises one or more circumferentially extending adhesive strips.

6. The implantable medical device of claim 1, wherein the barrier member comprises an impermeable membrane.

7. The implantable medical device of claim 1, wherein the barrier member comprises a silicone membrane or a polyurethane membrane.

8. The implantable medical device of claim 1, wherein the barrier member comprises a tissue matrix.

9. The implantable medical device of claim 1, wherein the barrier member comprises:
an inner layer;
an outer layer; and
a gel material disposed between the inner layer and the outer layer.

10. The implantable medical device of claim 9, wherein:
the inner layer is inelastic;
the outer layer is elastic; and
the gel material is reflowable.

11. A kit for treating a wound within a body lumen having an inner surface, the kit comprising:
an implant having a collapsed configuration and an expanded configuration, the implant including:
a barrier member having an outer surface; and
an adhesive pattern disposed on the outer surface of the barrier member; and
an expandable delivery element for delivering the implant, the expandable delivery element adapted to:
deliver the implant while the expandable delivery element and the implant are in a collapsed configuration; and
expand to cause the body lumen and the implant to expand, causing the adhesive pattern to contact the inner surface of the body lumen.

12. The kit of claim 11, wherein the expandable delivery element comprises an inflatable balloon.

13. The kit of claim 12, wherein the expandable delivery element comprises an inflatable balloon having a plurality of balloon folds when the inflatable balloon is deflated.

14. The kit of claim 13, wherein the barrier member is adapted to fold with the inflatable balloon such that the barrier member forms folds complementary to the balloon folds.

15. The kit of claim 11, wherein the adhesive pattern comprises an adhesive that is activated only after a period of time post-delivery.

16. The kit of claim 13, further comprising a sheath that is adapted to extend over the implant when the implant is in the collapsed configuration and to be withdrawn from the implant prior to expanding the expandable delivery element.

17. The kit of claim 13, wherein the body lumen comprises an esophagus.

18. A method for treating a wound within a body lumen, the method comprising:
advancing an assembly to the wound within the body lumen, the assembly comprising:
an inflatable balloon;
an implant disposed on the inflatable balloon and including a barrier member and an adhesive pattern disposed on the barrier member;
inflating the inflatable balloon in order to expand the barrier member and urge the adhesive pattern against an inner surface of the body lumen;
allowing the adhesive pattern to adhere to the inner surface of the body lumen; and
deflating the inflatable balloon.

19. The method of claim 18, wherein:
inflating the inflatable balloon also expands the body lumen; and
deflating the inflatable balloon allows the body lumen to return to a non-expanded configuration.

20. The method of claim 18, wherein the assembly further comprises a removable sheath extending over the implant, and the method further comprises:
withdrawing the removable sheath prior to inflating the inflatable balloon.
